(19) United States
(12) Patent Application Publication
(10) Pub. No.: US 2004/0228804 A1
(43) Pub. Date: Nov. 18, 2004

(54) Nasal administration of xylitol to a non-human mammal

(76) Inventors: Alonzo H. Jones, Plainview, TX (US); Jerry J. Bozeman, Plainview, TX (US)

Correspondence Address: CONLEY ROSE, P.C., P. O. BOX 3267, HOUSTON, TX, 77253-3267, US

(21) Appl. No.: 10/439,771

(22) Filed: May 16, 2003

(51) Int. Cl. A61L 9/04; A61K 31/045

(52) U.S. Cl. 424/45; 514/738

(57) ABSTRACT

A method of reducing or preventing the occurrence of respiratory infections, and other ailments associated with close quartering of non-human mammals such as cattle, the ailments generally referred to as “shipping fever.” The methods may comprise administering xylitol, possibly in the form a water or saline based solution, to the non-human mammals in a mist form. The mist may be applied directly to the nostrils of the animal, or may be applied in the general vicinity of the animal, such that the solution is inhaled.

### CROSS-REFERENCE TO RELATED APPLICATIONS

None.

### STATEMENT REGARDING FEDERALLY SPONSORED RESEARCH OR DEVELOPMENT

Not applicable.

### BACKGROUND OF THE INVENTION

1. Field of the Invention

Embodiments of the present invention are directed generally to medically treating non-human mammals, such as cattle. More particularly, embodiments of the invention are directed to treating illness in cattle associated with transportation and other close-quarter contact. Large animal veterinarians have ordinary skill in this art.

2. Background of the Invention

Cattle, like humans, are subject to becoming ill because of exposure to bacteria and viruses. Cattle on pasture land may experience fewer illnesses because, in part, of the limited population of other animals to which they are exposed. However, if these cattle come in contact with animals carrying bacteria and viruses to which they have not previously been exposed, illness based on the new strains is likely.

At times in the life cycle cattle, they may be moved from place to place, and in the process brought into contact with and exposed to different animals. More particularly, at some point cattle may be moved to a sale barn, where prior to the sale they are held in small pens and exposed to unfamiliar cattle. From the sale barn, cattle from disparate locations may combined into a group or lot and moved to other locations, like a feed lot or other pasture lands. During these movements, the cattle may be tightly packed in truck trailers. In fact, cattle are intentionally placed in the trucks in such a manner that the animals have very little room for movement, thus ensuring that no animals fall down where they may be subsequently trampled.

Cattle held in close-quarters, such as a group of cattle in a holding pen at a sale barn or within a trailer during shipping, tend to develop various illnesses thereafter. As an example, consider cattle shipped to a feed lot. On average, of cattle in the 500 to 600 pound range entering a feed lot, 25% percent may experience some type illness requiring treatment. Note that this is an average, and in some cases very little illness for a group of cattle may be experienced, yet in other cases an entire group of cattle may experience illness. Moreover, death loss, again on average, may be between 5% and 10%.

While there may be many reasons that cattle may become ill, such as contact with bacteria and virus to which they have not previously been exposed, there mere act of placing the cattle together for shipping (even a closed group of cattle) may cause many of the animals to develop illnesses such as fever associated with respiratory infections. The illnesses triggered by shipping of otherwise healthy cattle may be termed “shipping fever.” Shipping fever may account for half or more of the illness experience by cattle, and therefore may account for between 2.5% to 5% of the death loss of cattle entering a feed lot. At a price per animal of approximately $500 at this weight range, each lost animal may severely affect profitability.

Thus, what is needed is a method and related system to reduce illness in cattle associated with shipping and other close-quarter contact.

### BRIEF SUMMARY OF SOME OF THE PREFERRED EMBODIMENTS

Embodiments of the invention may be directed to reducing the occurrence of respiratory infections, and other illnesses generally referred as “shipping fever,” in non-human mammals such as cattle by administering xylitol. The xylitol may be delivered in a solution, and may be applied in mist form directly to one or more nostrils of the non-human animal. Alternatively, the solution comprising xylitol may be applied in mist form in the atmosphere surrounding the non-human animal.

The disclosed devices and methods comprise a combination of features and advantages which enable it to overcome the deficiencies of the prior art. The various characteristics described above, as well as other features, will be readily apparent to those skilled in the art upon reading the following detailed description.

### Notation and Nomenclature

Certain terms are used throughout the following description and claims to refer to particular system components, substances and features. This document does not intend to distinguish between components, substances and/or features that differ in name but not function.

In the following discussion and in the claims, the terms “including” and “comprising” are used in an open-ended fashion, and thus should be interpreted to mean “including, but not limited to . . . ”.

### DETAILED DESCRIPTION OF THE PREFERRED EMBODIMENTS

Embodiments of the invention are directed to methods for reducing shipping fever in non-human mammals, such as cattle. Because the methods were developed in the context of cattle, the following description is based on that developmental context; however, discussion of the various embodiments with respect to cattle should not be construed as limiting the methods disclosed herein to cattle only. These methods may be equally applicable to many non-human animals. Further, it should be understood however that the term “shipping fever” is not necessarily limited to illnesses associated with transportation of animals, but may also comprise illnesses associated with close quartering of animals, whether because of shipping constraints or space limitations in holding facilities.

The inventors of the present specification have discovered at least one cause of illness normally associated with the term “shipping fever,” and thus have also a devised methods to reduce or prevent such illness. When cattle are on pasture lands, or held in large areas, their normal stance is head prone. For example, as the cattle craze, or chew cud, their heads, including their nasal cavities, may be generally pointed downward. This head prone stance may allow nasal fluids, including bacteria in the nasal fluid, to exit by way of the nostrils.

In situations where the cattle are in close quarters, however, the head-prone stance may not be achievable. For example, in preparation for working the cattle or placing the cattle in trailers for shipping, the cattle may be held in a squeeze pen—a pen where very little movement of the cattle is possible. During shipping, cattle may be placed in the trailer in a close-packed manner which, as previously mentioned, may ensure that no animals fall, or lay down, and thus become subject to being trampled. However, holding the cattle together in this manner may also cause the cattle to hold their heads supine, allowing nasal fluid to run into the throat, and further allowing aspiration of the nasal fluid into the lungs. Nasal fluid may normally contain bacteria, but the problem may be exacerbated to some extent by feces and urine near the nose and mouth caused by the close quartering. Thus, ailments categorized as “shipping fever” may include fever associated with respiratory infections and pneumonia.

Embodiments of the present invention may reduce the occurrence of illness associated with shipping fever by treating the cattle with a solution comprising xylitol at one or more times including before shipping, during shifting, or after shipping. Alternative embodiments of the present invention may prevent or reduce the occurrence of shipping fever-like ailments associated with close quartering of cattle by treating the cattle with a solution comprising xylitol at one or more times including before, after and/or during the close quartering. Preferably, however, the xylitol may be administered after the trip.

Xylitol is the alcohol form of xylose, and is commonly used as a sweetener in food products. Xylitol also has the property that several pathogenic bacteria exposed to xylitol are less adherent to their environment. A co-inventor of the present specification holds a patent for the use of xylitol in solution for treating humans, U.S. Pat. No. 6,054,143 titled “Xylitol Delivery,” issued Apr. 25, 2000, incorporated by reference herein as if reproduced in full below, as well as patent for xylitol in solution, U.S. Pat. No. 6,258,372 titled “Xylitol Nose Spray,” issued Jul. 10, 2001, also incorporated by reference herein as if reproduced in full below.

Embodiments of the present invention are directed to treating cattle with xylitol in solution in order to reduce or prevent shipping fever, and/or ailments associated with close quartering of cattle. The solution may be a saline based solution for some embodiments, but the solution may also have just a water base. A maximum strength may be a saturated solution comprising approximately of 64 grams of xylitol per 100 cubic centimeters (cc's) of solution. The inventor has yet to determine a precise lower limit of xylitol that would be effective, but as little as 1% xylitol in solution has shown to be effective. It should be understood however, that a 1% xylitol in solution should not be construed as a lower limit of effectiveness; rather, this is only the lower limit of tested effectiveness. Smaller percentages of the xylitol in solution may likewise be effective, and thus their use would be within the scope and spirit of the invention.

The precise mode of application of the xylitol in solution may vary substantially. In at least some embodiments, nostrils of the cattle may be treated directly prior to close quartering, such as shipping. In particular, in these embodiments the xylitol in solution be applied to each nostril of each animal in the form of a mist (nebulized), which application preferably takes place for approximately two respiratory cycles of the animal for each nostril. In these embodiments, two to five fluid ounces (preferably four to five) total may be applied to each animal, approximately equally divided between the two nostrils. In yet other embodiments, the cattle may be treated by misting in each nostril for approximately two respiratory cycles after the close quartering has ended, e.g. after unloading the animals at their destination, such as a feed yard. Note that embodiments that pre-treat the animals, and the embodiments that post-treat the animals, are not mutually exclusive—the animals may be treated in this manner both before and after the close quartering situation. In these direct treatment embodiments, because of relatively low volumes of solution used as compared to other embodiments (discussed below), the solution may be a saline solution.

In yet other embodiments, the x-ylitol in solution may be applied to the animals during close quartering. For example, the xylitol may be administered during shipping, possibly within the shipping chamber such as a trailer. In the location of the close quartering, whether a cattle trailer for transportation or squeeze pen, the xylitol in solution may be misted or nebulized into the atmosphere near (preferably just above) the cattle, thus forcing the cattle to breath the solution. In the embodiments where the cattle are treated on a mass scale, a total amount of xylitol in solution nebulized preferably amounts to one-half pound of solution for each animal. For example, and without limitation, a set of fifty cows in a cattle trailer would preferably be misted with approximately 3 gallons of solution. Application of the solution in a mist may take place by a hand held device, similar to a yard chemical sprayer-type device. Alternatively, a mist system may be permanently or semi-permanently mounted in the area of the close quartering, e.g. within the cattle trailer and may be similar in form to water misting systems for cooling purposes. In the non-limiting example of a mist system in a cattle trailer, the cattle may be treated with xylitol in solution at any point in the journey, or at multiple points. Preferably, however, the xylitol in solution is applied at or near the destination.

The above discussion is meant to be illustrative of the principles and various embodiments of the present invention. Numerous variations and modifications will become apparent to those skilled in the art once the above disclosure is fully appreciated. For example, the precise time of application of the xylitol in solution may vary appreciably, and yet the benefits still obtained. Further, the various application methods may be used singularly, or in various combinations, to achieve the desired effect. Further still, while the specification has focused on the treatment of cattle, other mammals such as buffalo, horses, sheep, goats, dogs, cats and the like, may benefit treatments of xylitol in solution as well. Application volumes for this spectrum of animals may range from 0.005 cc's for small animals to 500 cc's for large animals. Finally, while the various embodiments have may use mists or nebulized solution, the xylitol in solution may likewise be applied in a purely aqueous form, and yet the benefits still obtained. It is intended that the following claims be interpreted to embrace all such variations and modifications.

What is claimed is:

1. A method comprising administering xylitol to a non-human mammal.

2. The method as defined in claim 1 further comprising administering the xylitol as a mist that enters through a nostril of the non-human mammal.

3. The method as defined in claim 2 further comprising administering the xylitol as a mist directly to a nostril of the non-human mammal.

4. The method as defined in claim 3 further comprising administering between approximately 0.005 cc to 500 cc of a solution comprising xylitol and water to the non-human mammal.

5. The method as defined in claim 3 further comprising administering a solution comprising xylitol and water to the nostril for approximately two respiratory cycles.

6. The method as defined in claim 2 further comprising administering a solution comprising xylitol and water as a mist in the atmosphere proximate to the non-human mammal.

7. The method as defined in claim 6 further comprising administering approximately one-half pound of the solution comprising xylitol and water in the atmosphere surrounding the non-human mammal.

8. A method of reducing the occurrence of shipping fever in cattle comprising administering xylitol to the cattle.

9. The method as defined in claim 8 further comprising administering the xylitol to each of the cattle on an individual basis before loading the cattle for shipping.

10. The method as defined in claim 8 further comprising administering the xylitol to each of the cattle on an individual basis after unloading from shipping.

11. The method as defined in claim 8 further comprising administering the xylitol to the cattle as a group within a shipping chamber.

12. The method as defined in claim 8 further comprising administering a solution containing xylitol to each of the cattle on an individual basis by applying the solution in mist form to a nostril of each of the cattle.

13. The method as defined in claim 12 wherein the solution containing xylitol comprises between approximately one and 64 parts xylitol, and 100 parts water.

14. The method as defined in claim 12 further comprising administering between four to five ounces of the solution containing xylitol to each of the cattle.

15. The method as defined in claim 8 further comprising administering a solution containing xylitol to the cattle by applying the solution in mist form to a shipping chamber.

16. The method as defined in claim 15 wherein the solution containing xylitol comprises between one and 64 parts xylitol, and 100 parts water.

17. The method as defined in claim 15 further comprising administering approximately one-half pound of the solution containing xylitol in the shipping chamber for each of the cattle.

18. A method comprising:
reducing an occurrence of shipping fever in cattle by;
administering a solution containing xylitol to the cattle.

19. The method as defined in claim 18 wherein the administering step further comprises administering the solution containing xylitol prior to shipping.

20. The method as defined in claim 18 wherein the administering step further comprises administering the solution containing xylitol just after arrival of the cattle at a destination.

21. The method as defined in claim 18 further comprising administering the solution containing xylitol in a mist form directly to a nostril of each of the cattle.

22. The method as defined in claim 21 further comprising administering a solution comprising between approximately one to sixty-four parts xylitol, and 100 parts water.

23. The method as defined in claim 21 further comprising administering between approximately four to five ounces to each of the cattle.

24. The method as defined in claim 18 further comprising administering the solution containing xylitol to the cattle in a shipping container by nebulizing the solution within the shipping chamber.

25. The method as defined in claim 24 further comprising nebulizing approximately one-half pound of the solution containing xylitol in the chamber for each of the cattle.

26. A method comprising reducing an occurrence of respiratory infection in cattle after close quartering of the cattle by administering a solution containing xylitol to the cattle.

27. The method as defined in claim 26 wherein the administering step further comprises administering the solution containing xylitol prior to the close quartering.

28. The method as defined in claim 26 wherein the administering step further comprises administering the solution containing xylitol just after the close quartering of the cattle has ended.

29. The method as defined in claim 26 further comprising administering the solution containing xylitol in a mist form directly to a nostril of each of the cattle.

30. The method as defined in claim 29 further comprising administering a solution comprising between approximately one to sixty-four parts xylitol, and 100 parts water.

31. The method as defined in claim 29 further comprising administering between approximately four to five ounces to each of the cattle.

32. The method as defined in claim 26 further comprising administering the solution containing xylitol to the cattle by misting the solution in the atmosphere proximate to the cattle.

33. The method as defined in claim 32 further comprising misting approximately one-half pound of the solution containing xylitol for each of the cattle.
